(19) United States
(12) Patent Application Publication
(10) Pub. No.: US 2004/0014241 A1
(43) Pub. Date: Jan. 22, 2004

(54) Method for analyzing bio-molecules

(75) Inventor: Sven Bulow, Hamburg (DE)

Correspondence Address: RANKIN, HILL, PORTER & CLARK, LLP, 700 HUNTINGTON BUILDING, 925 EUCLID AVENUE, SUITE 700, CLEVELAND, OH, 44115-1405, US

(73) Assignee: EPPENDORF AG, Hamburg (DE)

(21) Appl. No.: 10/454,185

(22) Filed: Jun. 4, 2003

(30) Foreign Application Priority Data
Jun. 5, 2002 (DE) 102 24 825.7

(51) Int. Cl. G01N 33/543

(52) U.S. Cl. 436/518

(57) ABSTRACT

A method for analyzing biomolecules. The biomolecules are immobilized on a support and then incubated with probe molecules that are capable of binding to the biomolecules. The probe molecules are provided with different markings, which comprise particle-shaped supports. The bound probe molecules are separated from the support and are analyzed using a separate apparatus, which is able to differentiate between the different markings.

The present invention relates to a method whereby, following immobilization on a substrate, the biomolecules are analyzed using marked probes specifically binding to these biomolecules.

A method of this kind for instance is the Reverse-Array-Technique described in EP 1,164,201. This technique immobilizes a target nucleic acid to be analyzed—which may be split into partial sequences—onto a substrate. Probes of oligonucleotides for instance fitted with fluorescent markings and each used with a different sequence are made to contact the immobilized target nucleic acid under hybridizing conditions. The unhybridized probes are removed by washing. Thereupon and subjected to denaturing conditions, the hybridized probes are separated from the target nucleic acid and made to contact an array on which a plurality of different oligonucleotides are immobilized each in a defined zone. The particle-shaped probes hybridize with the matching immobilized oligonucleotides on the array. A suitable screening device checks where in the array zones detectable markings can be detected. Hybridization with the marked oligonucleotides took place in those zones. It is known which oligonucleotides are configured in which array zones and a marking may be related to a given sequence which simultaneously corresponds to a partial sequence of the target nucleic acid. When using for instance combination theory in sequence analysis, the information gathered from the detected markings may lead to a longer segment or to the entire target sequence.

The known technique is fairly complex and moreover it is restricted to analyzing nucleic acid sequences.

Accordingly it is the objective of the present invention to further develop methods of the above kind so as to simplify them and also to make them more universally applicable.

This goal is attained by a method exhibiting the features of claim 1.

Accordingly the probe molecules used in the invention are fitted with different markings each of which may be directly detected in an appropriate apparatus. Within the scope of the present invention, detection and identification of the probe molecules must be implement using their markings.

Contrary to the case of the known methods, the invention requires for instance that each probe molecule exhibit its own marking which shall be detectable directly on account of an appropriate and in particular a physical apparatus and be related to the pertinent probe molecule, without introducing further biochemical or immunological reactions. The probe molecules used in the method of the present invention are known and as a result the detection of a given marking directly provides information about the biomolecule to be analyzed, for instance the presence of a particular sequence segment etc.

Directly detecting and identifying the probe molecule offers the considerable advantage that the method of the present invention is universally applicable. Unlike the above described Reverse-Array-Technique, it is NOT restricted to analyzing target nucleic acid molecules and to using oligonucleotides as the probe molecules.

On the other hand the method of the present invention allows analyzing all molecules for which there are specifically binding probe molecules. Illustratively proteins or peptides also may be immobilized to serve as molecules to be analyzed which shall then come into contact with antibodies, antibody fragments, with oligonucleotide-fitted antibodies, peptides, lectins, complex sugars, double-strand nucleic-acid molecules, RNA or specifically binding proteins acting as probe molecules.

To cite only one example, besides the already above stated sequences of oligonucleotides, also regulatory proteins may be used for instance to analyze nucleotide sequences.

The core of the invention consists in fitting the probe molecules with different, directly detectable markings which allow probe molecule identification without having to introduce a biochemical or immunological reaction.

In this invention, different probe molecules shall carry different markings. This definition is to be construed broadly and also allows for the possibility of several different probe molecules carrying the same marking and using combination techniques or other analytical methods to determine the actual or statistical association between the detected marking and the probes.

The particle-shaped marking strategy depends on the particular analytical goal. Especially well suited markings that may be used within the scope of the present invention will be inherently particular.

Conceivably the probe molecules may be linked with particles of different grain size, shape or different detectable dyes. The concept of “dye” is to be construed broadly. It shall include all detectable colorants, illustratively including fluorescent markings and invisible markings, for instance in the infrared range.

Such particular markings may be measured in especially simple manner for instance in a flow cytometer and be identified using grain size, shape and/or dyeing.

Quantom Dots for instance are illustrative of suitable markings and are comprehensively discussed in U.S. Pat. No. 6,207,392. They consist of fluorescent semiconductor nanocrystals and may be bound by linking molecules to the probe molecules.

Nano bar codes, which consist of latex beads bound to the probe molecules and which comprise a combination of fluorescent quantum dots of different colors as described in the above U.S. Pat. No. 6,207,392 also are appropriate.

Further appropriate bar codes consist of silicon particles which may coupled in covalent manner to the probe molecules and are marked with a machine-readable code that may be in the form of recesses, pits or crenelations or that may be coded by the very particle shape. Details relating to this bar code may be found in EP 0,863,797.

The “Luminex” beads illustratively described in U.S. Pat. No. 6,057,107 and consisting of a plurality of microspheres fitted with specific color codes and again may be coupled with the probe molecules are also appropriate.

The above discussion is not limitative. Instead all the particular markers which may be coupled to the probe molecules may be used, for instance those that may be identified and, as called for, quantified for instance in a flow detector or another directly detecting system.

The method of the present invention illustratively may be carried out in the manner of the Example described below.

### EXAMPLE

Toxicological test of a medicine's effect on rat liver cells

The rats are treated in a conventional manner with an agent of which the toxicity shall be ascertained. The measure of toxicity shall be the expression of genes of specific functional classes of which the expression products, in this instance RNA or cDNA copied therefrom during the test, are being determined. The same procedures are applied to an untreated control group.

One animal is removed at given times from the test and control groups. The animals are anesthetized and killed and their livers are prepared.

Following RNA extraction from the liver tissue using current purification procedures, the RNA of each rat liver is converted by reverse transcription into cDNA and is amplified by subsequent PCR. The PCR products are fed into reaction vessels with modified surface from accessible aldehyde groups. Following covalent cDNA binding by its amino groups to the vessel walls' aldehyde groups, free binding sites at the surfaces are then blocked.

Mixtures of probes are fed into each vessel, said probes recognizing representative genes of given relevant functional classes and being conjugated Luminex beads coded by a defined two-color marker signal. Illustratively the genes belong to the functional classes: apoptosis, cell cycle activation, cytochrom P450, DNA repairs, inflammation, glucuronyl transferase, oncogens, oxidizing stress, phosphorylation, senescence markers, transcription factors, transporters, TGF β receptors, tumor suppressors and tumor necrosis factors.

The probes are incubated in the reaction vessels under hybridization conditions. Following washing under stringent conditions to remove unbound particles, the bound particles are separated by raising the temperature and as a result they return into the reaction medium. The medium—which now contains the particles—is moved through a flow apparatus detecting the color code of each individual particle. In this manner, and solely on the basis of the particle-shaped particle code being present, in the absence of any further marking on the target molecule, binding events are detected and quantified.

Differences in the frequency of individual particle codes relating to specimens of untreated and treated rats allow ascertaining different expressions of the particular genes and hence the toxicological effects of the administered active ingredient. Moreover new active ingredients may be associated with known classes of mechanisms of operation, namely by comparing the expression pattern with those of medicines that were already investigated.

1. A method for analyzing biomolecules, wherein
(a) the biomolecules or parts thereof are immobilized onto a support,
(b) the support comprising the immobilized biomolecules then being incubated together with probe molecules which are able to specifically bind onto the biomolecules and which are fitted with a marking
(c) the unbound probe molecules being separated from the support comprising the probe molecules bound to it
(d) thereupon the bound probe molecules being separated from the support and being identified using a separate apparatus, and
(e) the information gathered in (d) about the probe molecules which were bound by the biomolecules in step b being analyzed,
the different probe molecules being fitted with different markings consisting of particle-shaped supports or being coupled to particle-shaped supports, said markings allowing directly identifying the probe molecule and the apparatus used in step (d) being able to differentiate between the different markings.

2. Method as claimed in claim 1, characterized in that the markings are particles and in that a particle detector is used for identification.

3. Method as claimed in claim 2, characterized in that the particles differ regarding their sizes and/or shapes and/or their codings with detectable colorants.

4. Method as claimed in one of claims 1-3, characterized in that the biomolecules are nucleic acid molecules.

5. Method as claimed in claim 4, characterized in that the probe molecules are single-strand oligonucleotides.

6. Method as claimed in claim 4, characterized in that the probe molecules are regulatory proteins.

7. Method as claimed in one of claims 1-3, characterized in that the biomolecules are proteins or peptides.

8. Method as claimed in claim 7, characterized in that the probe molecules are antibodies.

9. Method as claimed in either of claims 4 and 7, characterized in that the probe molecules are antibody fragments, antibodies fitted with oligonucleotides, peptides, lectins, complex sugars, double-strand nucleic acid molecules, RNA or specifically binding proteins.
